(19) United States
(12) Patent Application Publication
(10) Pub. No.: US 2014/0315936 A1
(43) Pub. Date: Oct. 23, 2014

(54) APRICITABINE AND PI COMBINATION THERAPY

(71) Applicants: Jonathan Alan Victor COATES, Mentone,Victoria (AU); Susan Wendy COX, Reservoir,Victoria (AU); AVEXA LIMITED, Richmond,Victoria (AU)

(72) Inventors: Jonathan Alan Victor Coates, Mentone (AU); Susan Wendy Cox, Reservoir (AU)

(21) Appl. No.: 14/359,023

(22) PCT Filed: Nov. 15, 2012
(86) PCT No.: PCT/AU2012/001409

§ 371 Date: May 16, 2014

(30) Foreign Application Priority Data
Nov. 18, 2011 (AU) 2011904831

(51) Int. Cl. A61K 31/513; A61K 45/06; A61K 31/4402

(52) U.S. Cl. CPC A61K 31/513; A61K 31/4402; A61K 45/06
USPC 514/274

(57) ABSTRACT

The present invention provides a method of treating HIV-1 infection in a subject. The method comprises administering to the subject a combination of anti-HIV-1 agents wherein the combination comprises an effective dose of Apricitabine and an effective dose of Atazanavir.

### FIELD OF THE INVENTION

The present invention relates to a combination of anti-viral agents to treat human immunodeficiency virus type 1 (HIV-1) infection. The combination includes Apricitabine and Atazanavir.

### BACKGROUND OF THE INVENTION

Since its discovery HIV-1 has been the subject of intense study to understand the virus and the pathogenesis associated with infection. These studies have lead to the development of a number of anti-HIV-1 agents which target different points in the HIV-1 replication cycle and in the manner in which the virus infects cells. These agents fall into the following groups: (i) nucleoside and nucleotide reverse transcriptase inhibitors (NRTI); (ii) nonnucleoside reverse transcriptase inhibitors (NNRTI); (iii) protease inhibitors (PI); (iv) integrase inhibitors (INSTI); and (v) binding and entry inhibitors. Examples of these agents and the target of their activity are set out in Table 1.

TABLE 1
Anti-HIV-1 Agents

| Compound type and generic | Common | |
|---|---|---|
| name | abbreviation | Target |
| NRTIs | | |
| Azidothymidine | AZT | HIV-1 reverse transcriptase |
| Zalcitabine | ddC | |
| Didanosine | ddI | |
| Lamivudine | 3TC | |
| Stavudine | D4T | |
| Abacavir | ABC | |
| Emtricitabine | FTC | |
| Tenofovir | TDF | |
| Apricitabine | ATC | |
| NNRTIs | | |
| Nevirapine | NVP | HIV-1 reverse transcriptase |
| Delaviridine | DLV | |
| Efavirenz | EFV | |
| Etravirine | ETV | |
| Rilpivarine | RPV | |
| PIs | | |
| Amprenavir | APV | HIV-1 protease |
| Indinavir | IDV | |
| Nelfinavir | NFV | |
| Atazanavir | ATV | |
| Lopinavir | LPV | |
| Saquinavir | SQV | |
| Darunavir | DRV | |
| Fosamprenavir | FPV | |
| Ritonavir | RTV | |
| Tipranavir | TPV | |
| INSTI | | |
| Raltegravir | RAL | HIV-1 integrase |
| Binding and entry inhibitors | | |
| Maraviroc | MVC | CCR5 in its role as an HIV- |
| | | coreceptor |
| Enfuvirtide | T-20 | gp-41-mediated fusion |

It has long been recognized mono-therapy for HIV-1 infection is only transiently effective. This has lead to the development of combination therapies for HIV-1 (HAART). The principle driving force behind the development of these therapies was the emergence of drug resistance following the therapeutic use of single antiretroviral drugs such as AZT.

Apricitabine (4-amino-1-[(2R,4R)-2-(hydroxymethyl)-1,3-oxathiolan-4-yl]pyrimidin-2(1H)-one((ATC) is a nucleoside reverse transcriptase inhibitor (NRTI) active against HIV. It is structurally related to Lamivudine and Emtricitabine, and, like these, is an analogue of cytidine.

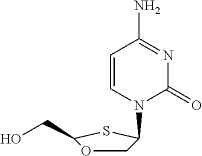 [SMILES: Nc1ccn([C@H]2CO[C@@H](CO)S2)c(=O)n1]

### Apricitabine

Atazanavir (methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) (ATV), marketed under the trade name Reyataz by Bristol Myers, is an antiretroviral drug of the protease inhibitor (PI) class. Atazanavir is distinguished from other PIs in that it can be given once-daily (rather than requiring multiple doses per day) and has lesser effects on the patient's lipid profile. Like other protease inhibitors, it is used only in combination with other HIV medications.

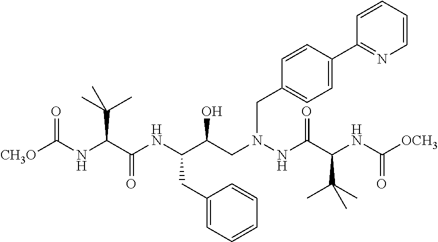

### Atazanavir

### SUMMARY OF THE INVENTION

The present inventors have found that a combination of Apricitabine and Atazanavir is more efficacious than a combination of Lamivudine and Atanazavir.

Accordingly, a first aspect the present invention provides a method of treating HIV-1 infection in a subject comprising administering to the subject a combination of anti-HIV-1 agents wherein the combination comprises an effective dose of Apricitabine and an effective dose of Atanazavir.

In a second aspect the present invention provides a combination of anti-HIV-1 agents wherein the combination comprises an effective dose of Apricitabine and an effective dose of Atanazavir for treating HIV-1 infection.

### DETAILED DESCRIPTION OF THE INVENTION

The present invention relates to combination therapies for HIV-1 infection. As mentioned above the present inventors have found that a combination of ATC and ATV is more useful in reducing viral load than a combination of 3TC and ATV. Accordingly the present invention relates to combination therapy where the combination comprises ATC and ATV.

The combination comprises an effective dose of both ATC and ATV. As used herein an “effective dose” is a dose which results in a reduction of viral load. Typically an effective dose of ATC is 400 to 1200 mg b.i.d and an effective dose of ATV is 400 mg QD or 300 mg QD with 100 mg Ritonavir QD.

It is preferred that the combination includes one or more other anti-HIV-1 agents. Preferably the combination includes NRTIs such as Azidothymidine, Didanosine, Lamivudine, Stavudine, Abacavir; NNRTIs such as Nevirapine, Delaviridine, Efavirenz Rilpivirine and Etravirine; the NtRTI Tenofovir: PI's such as Amprenavir, Indinavir, Nelfinavir, Lopinavir, Saquinavir, Darunavir, Fosamprenavir, Tipranavir and Ritonavir; INSTIs such as Raltegravir and the Binding and entry inhibitors such as Maraviroc and Enfuvirtide.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

As used in the subject specification, the singular forms “a”, “an” and “the” include plural aspects unless the context clearly dictates otherwise. Thus, for example, reference to “a” includes a single as well as two or more; reference to “an” includes a single as well as two or more; reference to “the” includes a single as well as two or more and so forth.

Having generally described the invention, the same will be more readily understood by reference to the following examples, which are provided by way of illustration and are not intended as limiting.

### EXAMPLES OF THE INVENTION

Groups of HIV-1 positive patients were treated (as part of an optimized regimen) with ATC+either LPV or ATV, and compared to patients treated with 3TC+either LPV or ATV. Samples were taken after therapy to determine the number of patients who achieved an undetectable plasma HIV-1 RNA level (<50 copies/mL). The results are set out in Table 2.

TABLE 2

| Patients achieving <50 copies/mL | |
|---|---|
| 800 mg BID ATC | 150 mg BID 3TC |

| Drug | n/N | % | n/N | % |
|---|---|---|---|---|
| LPV | 18/30 | 60 | 18/29 | 62 |
| ATV | 8/12 | 66.7 | 6/14 | 42.9 |

A surprisingly greater number of patients receiving ATC+ATV responded compared to those receiving 3TC+ATV.

1. A method of treating HIV-1 infection in a subject comprising administering to the subject a combination of anti-HIV-1 agents wherein the combination comprises an effective dose of Apricitabine and an effective dose of Atazanavir.

2. A method as claimed in claim 1 wherein the combination further comprises at least one additional anti-HIV agent selected from the group consisting of Azidothymidine, Didanosine, Lamivudine, Stavudine, Abacavir, Nevirapine, Delaviridine, Efavirenz, Rilpivirine, Etravirine, Tenofovir, Amprenavir, Indinavir, Nelfinavir, Lopinavir, Saquinavir, Darunavir, Fosamprenavir, Tipranavir, Ritonavir, Raltegravir, Maraviroc, Enfuvirtide and combinations thereof.

3. A combination of anti-HIV-1 agents wherein the combination comprises an effective dose of Apricitabine and an effective dose of Atazanavir for treating HIV-1 infection.

4. A combination as claimed in claim 3 wherein the combination further comprises at least one additional anti-HIV agent selected from the group consisting of Azidothymidine, Didanosine, Lamivudine, Stavudine, Abacavir, Nevirapine, Delaviridine, Efavirenz, Rilpivirine, Etravirine, Tenofovir, Amprenavir, Indinavir, Nelfinavir, Lopinavir, Saquinavir, Darunavir, Fosamprenavir, Tipranavir, Ritonavir, Raltegravir, Maraviroc, Enfuvirtide and combinations thereof.
